(19) United States
(12) Patent Application Publication
(10) Pub. No.: US 2012/0220879 A1
(43) Pub. Date: Aug. 30, 2012

(54) Catheter and Catheter Arrangement

(75) Inventors: Stephan Fandrey, Affoltern am Albis (CH); Michael Diebold, Berlin (DE)

(73) Assignee: VascoMed GmbH, Binzen (DE)

(21) Appl. No.: 13/369,965

(22) Filed: Feb. 9, 2012

Related U.S. Application Data
(60) Provisional application No. 61/446,053, filed on Feb. 24, 2011.

(51) Int. Cl. A61B 6/00

(52) U.S. Cl. 600/478

(57) ABSTRACT

A catheter, in particular an ablation catheter, comprising force sensors integrated in a distal section, which are designed and disposed to measure the magnitude and direction of an external force acting on the distal section, and which can be connected to a signal processing unit for the combined processing of measurement signals, wherein a single FBG fiber fastened in a sensor holder is provided in the catheter and comprises three force sensor regions forming the force sensors which interact for the combined processing of measurement signals.

### CROSS-REFERENCE TO RELATED APPLICATIONS

This patent application claims the benefit of co-pending U.S. Provisional Patent Application No. 61/446,053, filed on Feb. 24, 2011, which is hereby incorporated by reference in its entirety.

### TECHNICAL FIELD

The present invention relates to a catheter and, in particular, to an ablation catheter, comprising force sensors integrated in a distal section, which are designed and disposed to measure the magnitude and direction of an external force acting on the distal section, and which can be connected to a signal processing unit for the combined processing of measurement signals.

In certain fields of application of catheters or similar devices, e.g., electrode leads, a contact force on adjacent tissue is significant for the functionality thereof and, therefore, detection of this contact force is of interest. This is, for example, particularly significant for ablation catheters, which are used to ablate tissue regions or to remove tissue parts.

### BACKGROUND

An ablation catheter (for example, a “TactiCath”, from the company Enclosense) is known, which permits the measurement of a force acting on the distal end of the catheter—which is the aforementioned contact force in this application—in terms of magnitude and direction during an ablation procedure. This catheter uses the principle of the FBG (Fiber Bragg Grating) sensor, in which case three fibers each having an FBG sensor on the fiber end form the group of sensors required for a 3D force measurement, and which can be connected to a signal processing unit for the combined processing of measurement signals. The sensors are installed externally on a deformable cylinder with 120° of separation between them.

U.S. Publication No. 2008/0285909 provides an extensive description of the mode of operation of FBG sensors for the determination of transpositions or curvatures of the catheter body, as well as an explanation of the mode of operation of the aforementioned force sensor having a plurality of FBG fibers on a deformable cylinder.

As described in International Publication No. WO 2009/138957, temperature compensation using three electrical thermal elements is provided, because in the FBG measurement process even slight temperature changes or deviations between the individual sensors can cause great measurement uncertainties and, in the case of an electrothermal ablation procedure, very substantial temperature fluctuations can occur on the tip of the ablation catheter.

The optical measurement principle of the FBG sensor system is known in general and, in particular, in regard to the application thereof for force and temperature measurements. See, e.g., “www.wikipedia.org/wiki/Fiber_Bragg_grating”, and also (especially in regard to stress and temperature measurements) U.S. Pat. No. 5,399,854. An extensive explanation of the measurement principle is therefore not required.

Independently of this measurement principle, other solutions are also known for measuring contact force on a guide wire or catheter, e.g., using an optical sensor, as described in International Publication No. WO 2009/007857, or using a semiconductor sensor on the tip of a guide wire, as described in International Publication No. WO 2008/003307.

A problem to be solved by the present invention is that of providing a catheter of the originally stated type that is simplified in terms of the design thereof and the connection to an associated signal processing unit, and which is therefore more economical.

The present inventive disclosure is directed toward overcoming one or more of the above-identified problems.

### SUMMARY

This problem is solved by a catheter having the features of certain independent claim(s). Furthermore, a corresponding catheter system of that type having the features of certain other independent claim(s) is provided, as well as a novel force-measuring device having the features of yet certain other independent claim(s). Advantageous developments of the inventive disclosure are the subject matter of the respective dependent claims.

The present invention incorporates the main aspect of providing a single FBG fiber in a catheter having force sensors for the 3D measurement of force, with the single FBG fiber comprising the force sensor regions required to detect the magnitude and direction of the external force (especially contact force). It also incorporates the aspect of fastening these individual fibers in a sensor holder in a manner such that the force sensor regions can function as intended in/on the fiber. The force-measuring device according to the present invention basically has the same features.

In particular, the present invention therefore provides a contact force sensor which has a simple design and is integrated in a catheter tip, and which can measure the contact force between the catheter tip and the tissue wall, especially during ablation. This ultimately makes it possible to establish defined contact between an ablation electrode on the catheter tip and the vascular wall, thereby reliably ensuring that the success of the ablation procedure is satisfactory. The solution according to the present invention also makes it possible to use a simpler and more economical signal processing unit, since the sensors disposed on an individual fiber can be read out via a single channel.

In particular, the three force sensor regions are formed on the FBG fiber such that they are interspaced in the longitudinal direction thereof.

In one embodiment of the present invention, the FBG fiber is situated closely against a cooling channel of the catheter such that, during operation, the force sensor regions record a temperature that is close to the temperature of a cooling or rinse fluid conducted through the cooling or rinse channel. By way of this embodiment, the temperature of the sensor regions is stabilized, and therefore external temperature changes (e.g., during an ablation procedure) affect the sensor system only slightly, and any additional elements required for temperature measurement or compensation can be omitted.

In one configuration of this embodiment, the rinse channel is shaped as a helix, at least in one section of the catheter, in which the force sensor regions are formed on the FBG fiber, and the FBG fiber extends along or close to the longitudinal axis of the helix.

In a further embodiment of the present invention, a temperature sensor is disposed in the rinse channel in order to provide a temperature reference value that very closely approximates the actual temperature of the sensor regions. This sensor makes it possible for the temperature along the length of the fiber grating (sensor regions) for force measurement to be known and derived by way of the rinse fluid, and temperature compensation can be carried out on the basis of a catheter-specific calibration matrix.

It is also within the scope of the above-described embodiment and configurations to accommodate the FBG fiber in the catheter body such that it is centered to the greatest extent possible at a great distance from the outer wall of the catheter.

In a further embodiment of the present invention, temperature sensor regions are formed on the FBG fiber close to the force sensor regions and, in particular, there between. This permits a computer-assisted temperature compensation of the signals of the individual force sensor regions to be carried out, in particular, in applications having strong temperature fluctuations or in structural embodiments where a deviating constancy of temperature of the force sensor regions cannot be attained by the rinse fluid. The temperature sensor regions should be free of stress, mechanical stress in particular, in the sensor holder mentioned above, to prevent the temperature signals from becoming corrupted by mechanical loads on the fiber.

In a further embodiment of the present invention, a temperature sensor region is formed on the distal end of the FBG fiber. A temperature measurement on the catheter end is desired in the case of ablation catheters, in particular, and they can be performed in conjunction with the force measurement of a single fiber in the above-mentioned embodiments. In turn, this results in a simpler design of the associated signal processing unit and the connection thereto.

Within the scope of these simplifications, a further embodiment provides that the individual force sensor regions and, in particular, the optionally provided temperature sensor regions for operation using different wavelengths is designed such that a readout can be performed via a single input channel of the signal processing unit.

In a further embodiment of the present invention, the sensor holder comprises structurally predetermined, mechanical weak points corresponding to the position of the force sensor regions, in particular, in the form of a helically extending recess or a plurality of recesses having the shape of a circular segment, in a cylindrical holder housing. As a result, the FBG fiber is mechanically protected, and the optionally provided temperature sensor regions can be mechanically relieved as necessary. This design also ensures that the force sensor regions, which basically function as strain-measuring regions, can function as intended.

Considering that the knowledge of sensor-specific or catheter-specific data of the individual product in an enclosed evaluation unit is required to measure force with great accuracy, the catheter according to a further embodiment of the present invention comprises an information carrier which has specific calibration information and is disposed, in particular, on or close to a proximal end. In one embodiment, the information carrier is designed, for example, as an EEPROM, RFID tag, or barcode, which can be read out easily and reliably using means on the signal processing unit that are known per se and are economical.

According to one embodiment of the catheter system that is provided, the signal processing unit comprises an input channel for signals from temperature sensor regions and/or a temperature sensor in the rinse channel of the catheter, and a temperature calculation unit and/or compensation processing means for the temperature compensation of signals of the force sensors. In particular, a common input channel is provided for signals of the force sensors and the temperature sensor regions.

According to a further embodiment of the arrangement, the signal processing unit comprises readout means for reading out calibration information from an information carrier on the catheter.

Embodiments of the proposed force-measuring device basically result from the above-described embodiments of the proposed catheter, provided they do not refer to special features of the catheter (such as, for example, the rinse channel or the temperature measurement on the catheter end).

Advantages and useful features of the invention also result from the description that follows of embodiments with reference to the Figures.

Various other objects, aspects and advantages of the present inventive disclosure can be obtained from a study of the specification, the drawings, and the appended claims.

### DESCRIPTION OF THE DRAWINGS

The inventive subject matter will be described in greater detail in the following using preferred embodiments, with reference to the drawings and the reference characters noted therein. In the drawings:

FIG. 1 shows a schematic diagram of the design of an FBG fiber;

FIG. 2 shows a schematic diagram of the sensor design in an embodiment of the catheter according to the present invention;

FIG. 3 shows a schematic diagram of an embodiment of the catheter according to the present invention, comprising a specially designed rinse channel; and

FIGS. 4A and 4B show schematic diagrams of the design of the sensor holder in two further embodiments of the catheter according to the present invention.

### DETAILED DESCRIPTION

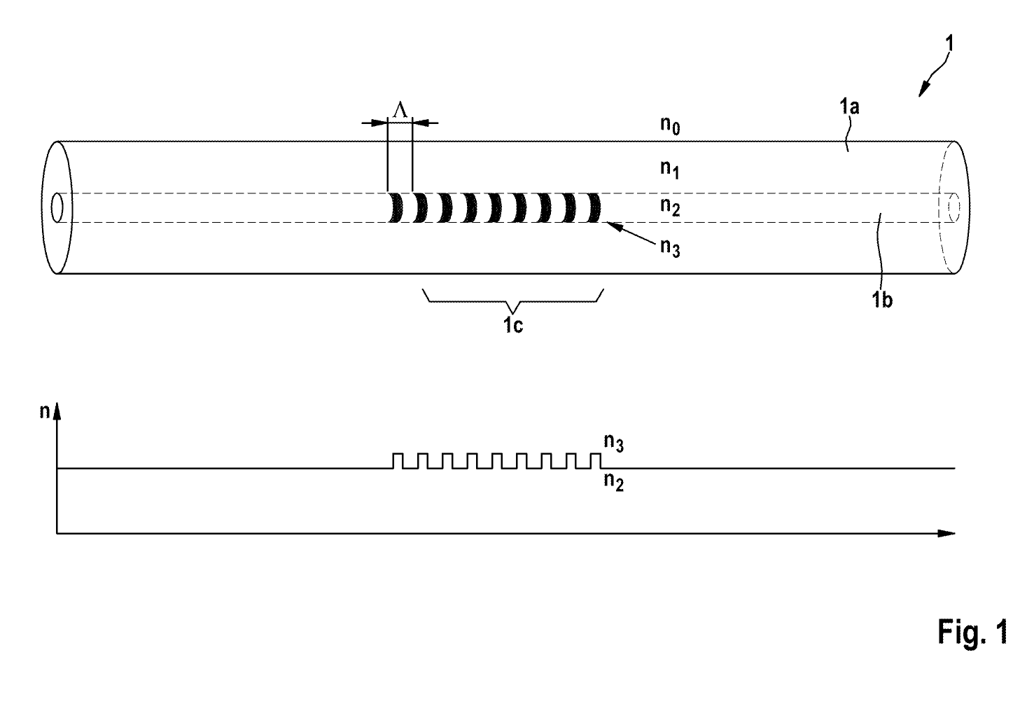

FIG. 1 shows, at the top and as a schematic perspective depiction, a section of an FBG fiber 1 having a core 1b which has a refractive index of n2 substantially along the entire longitudinal extension, and which is embedded in a jacket 1a having a refractive index n1. In one sensor section 1c of fiber 1, a Bragg grating is formed, in which sections having refractive index n2 alternate with sections of another refractive index n3, as illustrated in the bottom part of FIG. 1. Sensor section 1c of fiber 1 can be used, among other things, to detect stress or applications of external force or temperature changes, as is known from the prior art, and will therefore not be described in greater detail here.

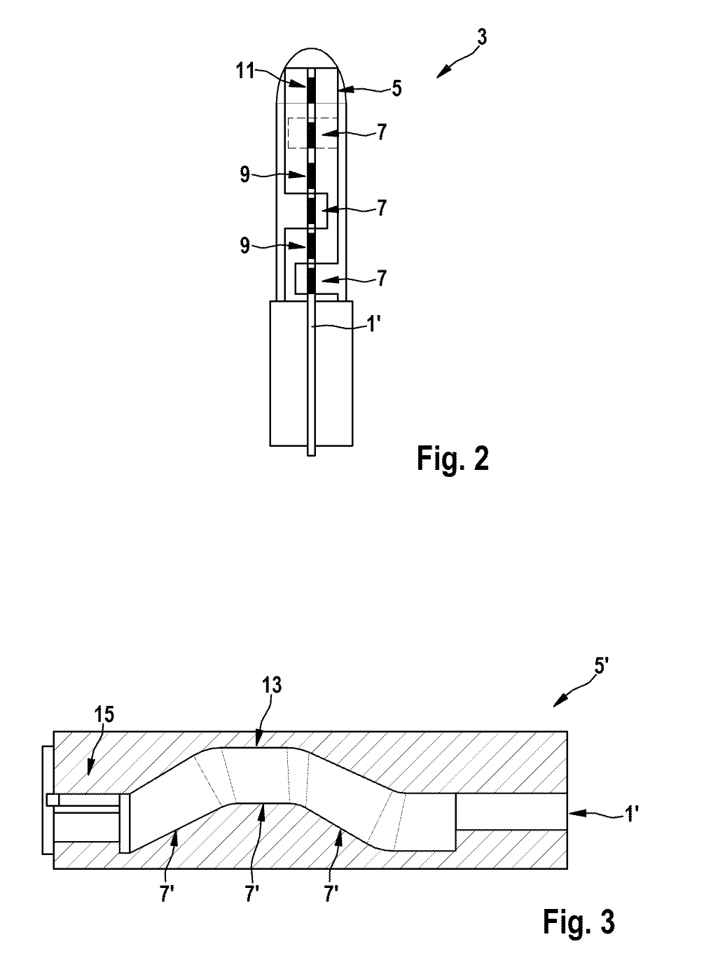

FIG. 2 shows, as a schematic diagram, a distal section of an ablation catheter 3, in the center of which an FBG fiber 1′ extends and is held in a sensor holder 5. A plurality of sensor regions having different functions are formed in FBG fiber 1′. In particular, FIG. 2 shows three force sensor and strain sensor regions 7, two temperature sensor regions 9 situated there between, the signals of which are used for the temperature compensation of signals from force sensor regions 5, and a further temperature sensor region 11 on the distal end of fiber 1′ for determining the temperature of the electrode tip during use, i.e., during an ablation procedure. Individual FBG sensor regions 7, 9 and 11 can be manufactured such that each one on fiber 1′ (by way of which the signals are transmitted simultaneously, due to the design) is designed to utilize a different wavelength. Each sensor region can therefore be read out independently.

The temperature sensor regions provided between the force sensor regions deliver signals which can be used for temperature calibration if temperature stabilization of the distal catheter section is inadequate.

Furthermore, the sensor system must be calibrated in order to calculate a 3D force vector, by loading the sensor with three orthogonal forces (Fx, Fy, Fz) in succession.

In theory, strains ε₁, ε₂ and ε₃ in the distributed sensor regions are calculated with bending resistances EI, bending moments Fl, and distance r to the strain-neutral fiber, as follows (see FIG. 1):

$${ɛ}_{1} = \frac{{F}_{x} \cdot {l}_{1}}{E \cdot {I}_{xx 1}} \cdot {r}_{11} + \frac{{F}_{y} \cdot {l}_{1}}{E \cdot {I}_{yy 1}} \cdot {r}_{12} + \frac{{F}_{z}}{A \cdot E} \\ {ɛ}_{2} = \frac{{F}_{x} \cdot {l}_{2}}{E \cdot {I}_{xx 2}} \cdot {r}_{21} + \frac{{F}_{y} \cdot {l}_{s}}{E \cdot {I}_{yy 2}} \cdot {r}_{22} + \frac{{F}_{z}}{A \cdot E} \\ {ɛ}_{3} = \frac{{F}_{x} \cdot {l}_{3}}{E \cdot {I}_{xx 3}} \cdot {r}_{31} + \frac{{F}_{y} \cdot {l}_{3}}{E \cdot {I}_{yy 3}} \cdot {r}_{32} + \frac{{F}_{z}}{A \cdot E} \qquad Eq . 1$$

A calibration matrix can be calculated by performing three measurements, in which three orthogonal forces are used, as follows:

$$( \begin{matrix}{C}_{11} & {C}_{12} & {C}_{13} \\ {C}_{21} & {C}_{22} & {C}_{23} \\ {C}_{31} & {C}_{32} & {C}_{33}\end{matrix} ) = ( \begin{matrix}{F}_{x} & 0 & 0 \\ 0 & {F}_{y} & 0 \\ 0 & 0 & {F}_{z}\end{matrix} ) \cdot {( \begin{matrix}{ɛ}_{11} & {ɛ}_{12} & {ɛ}_{13} \\ {ɛ}_{21} & {ɛ}_{22} & {ɛ}_{23} \\ {ɛ}_{31} & {ɛ}_{32} & {ɛ}_{33}\end{matrix} )}^{- 1} \qquad Eq . 2$$

In that case, bending resistances EI and distances to neutral fiber r in the sensor holder must be designed such that three linearly independent equations result. It is thus possible to calculate a 3D force vector from strains measured from any force direction using the calibration matrix:

$$( \begin{matrix}{F}_{x} \\ {F}_{y} \\ {F}_{z}\end{matrix} ) = ( \begin{matrix}{C}_{11} & {C}_{12} & {C}_{13} \\ {C}_{21} & {C}_{22} & {C}_{23} \\ {C}_{31} & {C}_{32} & {C}_{33}\end{matrix} ) \cdot ( \begin{matrix}{ɛ}_{1} \\ {ɛ}_{2} \\ {ɛ}_{3}\end{matrix} ) \qquad Eq . 3$$

These calibration data are optionally stored in the catheter on, for example, an EEPROM, RFID or a 2D barcode, and are read out from the evaluation unit wirelessly, in a wired manner, or optically in the case of a 2D barcode.

FIG. 3 shows a sensor holder 5′ of a catheter according to the present invention, in the case of which a rinse channel 13 in the distal section, where three force sensor regions 7′ are disposed on FBG fiber 1′, is designed such that it encloses the fiber in a helical manner. Fiber channel 15, as such, is designed as a straight line. The helical design of rinse channel 13 around fiber channel 15 is designed to ensure that the temperature of sensor regions 7′ remains relatively constant even in applications in which temperatures fluctuate greatly (as, for example, in ablation). In addition, the special design of the helical rinse channel 13 ensures that the three force sensor regions are linearly independent.

Table 1 below shows the results of a simulation calculation for sensor holder 5′, which is based on the algorithm illustrated above, and on the basis of which the functionality of sensor holder 5′ can be derived.

TABLE 1
Example results of a simulation of the sensor holder according to FIG. 3

| Force introduced | Simulated strain of the | Force vector calculated |
|---|---|---|
| in the simulation | three sensor regions | from the simulated strains |
| Fx = −0.25 N | ε₁ = 96 μstrain | Fx = −0.2602 N |
| Fy = −0.25 N | ε₂ = 116 μstrain | Fy = −0.255 N |
| Fz = 0 N | ε₃ = 64 μstrain | Fz = −0.006 N |

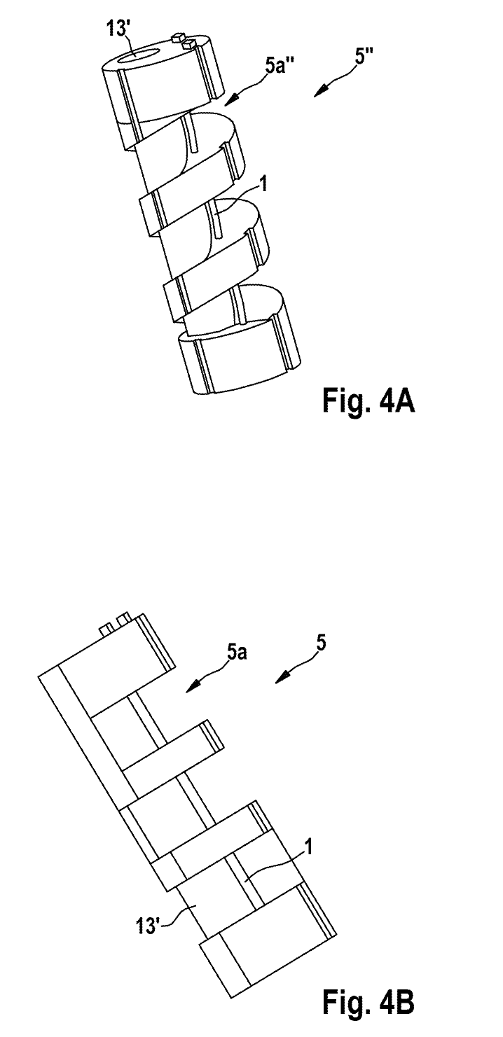

FIG. 4A shows a further sensor holder 5″ which comprises a rinse channel 13′ (which extends in a straight line in this case), as well as FBG fiber 1 (which likewise extends in a straight line), and a helically extending recess 5a″ as a weakening region. By way of the latter, it is ensured that force can act largely freely on the force sensor regions (which are not depicted here) on fiber 1.

Table 2 below shows exemplary results of a simulation calculation for this sensor holder 5″.

TABLE 2
Example results of a simulation of the sensor holder of FIG. 4A.

| Force introduced | Simulated strain of the | Force vector calculated |
|---|---|---|
| in the simulation | three sensor regions | from the simulated strains |
| Fx = 0.35 N | ε₁ = 59 μstrain | Fx = 0.3484 N |
| Fy = −0.125 N | ε₂ = 210 μstrain | Fy = −0.120 N |
| Fz = −0.2 N | ε₃ = 406 μstrain | Fz = −0.1919 N |

As a further embodiment, FIG. 4B shows a perspective depiction of sensor holder 5, which is also shown in FIG. 2 as a sketch. In this method as well, rinse channel 13′ and FBG fiber 1 extend closely adjacent to one another and in a straight line. Weakening regions 5a are embodied in this case as segments of circular rings which are offset by approximately 120° and are separated by recesses, in order to ensure linear independence of the sensors in this case as well. Table 3 below shows simulation results for this sensor holder 5.

TABLE 3
Example results of a simulation of the sensor holder of FIG. 4B.

| Force introduced | Simulated strain of the | Force vector calculated |
|---|---|---|
| in the simulation | three sensor regions | from the simulated strains |
| Fx = 0.35 N | ε₁ = 37 μstrain | Fx = 0.35002 N |
| Fy = −0.125 N | ε₂ = 224 μstrain | Fy = −0.1249 N |
| Fz = −0.2 N | ε₃ = 281 μstrain | Fz = −0.19997 N |

The embodiments of the present invention is not limited to the above-described examples and emphasized aspects but, rather, are possible in a large number of modifications that lie within the scope of handling by a person skilled in the art.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. A catheter comprising:
force sensors integrated in a distal section, which are designed and disposed to measure the magnitude and direction of an external force acting on the distal section, and which can be connected to a signal processing unit for the combined processing of measurement signals, wherein a single FBG fiber fastened in a sensor holder is provided in the catheter and comprises three force sensor regions forming the force sensors which interact for the combined processing of measurement signals.

2. The catheter of claim 1, wherein the catheter comprises an ablation catheter.

3. The catheter according to claim 1, wherein the three force sensor regions are formed on the FBG fiber such that they are interspaced in a longitudinal direction thereof.

4. The catheter according to claim 1, wherein temperature sensor regions are formed on the FBG fiber close to the force sensor regions.

5. The catheter according to claim 1, wherein temperature sensor regions are formed on the FBG fiber between the force sensor regions.

6. The catheter according to claim 1, wherein a temperature sensor region is formed on the distal end of the FBG fiber.

7. The catheter according to claim 1, wherein the FBG fiber is situated closely against a cooling channel of the catheter such that, during operation, the force sensor regions assume a temperature that is close to the temperature of a cooling or rinse fluid conducted through the rinse channel.

8. The catheter according to claim 7, wherein the cooling channel is shaped as a helix, at least in one section of the catheter, in which the force sensor regions are formed on the FBG fiber, and the FBG fiber extends along or close to a longitudinal axis of the helix.

9. The catheter according to claim 7, wherein a temperature sensor is disposed in the cooling channel.

10. The catheter according to claim 1, wherein the sensor holder comprises structurally predetermined, mechanical weak points corresponding to the position of the force sensor regions, in the form of a helically extending recess or a plurality of circular recesses in a cylindrical holder housing.

11. The catheter according to claim 1, wherein temperature sensor regions are formed on the FBG fiber close to the force sensor regions, wherein the individual force sensor regions and the temperature sensor regions are designed to operate using different wavelengths, such that a readout via a single input channel of the signal processing unit is possible.

12. The catheter according to claim 1, further comprising one information carrier which has specific calibration information which is disposed on or near a proximal end.

13. The catheter according to claim 12, wherein the information carrier is designed as an EEPROM, RFID tag, or barcode.

14. A catheter system comprising:
a catheter comprising:
force sensors integrated in a distal section, which are designed and disposed to measure the magnitude and direction of an external force acting on the distal section, and which can be connected to a signal processing unit for the combined processing of measurement signals, wherein a single FBG fiber fastened in a sensor holder is provided in the catheter and comprises three force sensor regions forming the force sensors which interact for the combined processing of measurement signals; and
a signal processing unit connected to the catheter and which comprises a single input channel for the signals of the force sensors.

15. The catheter system according to claim 14, wherein the catheter comprises an ablation catheter.

16. The catheter system according to claim 14, wherein the signal processing unit comprises an input channel for signals from temperature sensor regions or a temperature sensor in a cooling channel of the catheter, or both, and a temperature calculation unit or compensation processing means for the temperature compensation of signals of the force sensors, or both.

17. The catheter system according to claim 14, wherein a common input channel for signals of the force sensors and temperature sensor regions is provided.

18. The catheter system according to claim 14, wherein the signal processing unit comprises readout means for reading out calibration information from an information carrier on the catheter.

19. A force measurement device for measuring the magnitude and direction of a force acting on a mechanical part, comprising:
a single FBG fiber fastened in a sensor holder wherein the single FBG fiber comprises three force sensor regions forming force sensors which interact for the combined processing of measurement signals.

20. The force measurement device according to claim 19, wherein the three force sensor regions are formed on the FBG fiber such that they are interspaced in a longitudinal direction thereof.

21. The force measurement device according to claim 19, wherein the sensor holder comprises structurally predetermined, mechanical weak points corresponding to the position of the force sensor regions, in the form of a helically extending recess or a plurality of recesses having a shape of a circular segment, in a cylindrical holder housing.

22. The force measurement device according to claim 19, wherein the individual force sensor regions are designed to operate using different wavelengths such that a readout via a single input channel of a signal processing is possible.
